(19) United States
(12) Patent Application Publication
(10) Pub. No.: US 2026/0263734 A1
(43) Pub. Date: Sep. 10, 2026

(54) SLEEP STATE INFLUENCE THROUGH DISPENSING ACTIVE COMPOUNDS

(71) Applicant: Nicoventures Trading Limited, London (GB)

(72) Inventors: Jeffrey Sean SMITH, London (GB); Sean LUKAN, London (GB); Laura SMITH, London (GB); Chloe CORDERY, London (GB)

(21) Appl. No.: 19/165,239

(22) PCT Filed: Mar. 15, 2024
(86) PCT No.: PCT/GB2024/050700

§ 371 Date: Sep. 15, 2025

Related U.S. Application Data
(63) Continuation of application No. 18/184,275, filed on Mar. 15, 2023, now abandoned.

(51) Int. Cl. A61M 21/02; A61M 21/00

(52) U.S. Cl. CPC A61M 21/02; A61M 2021/0077; A61M 2230/18

(57) ABSTRACT

An apparatus, method and computer program is described comprising: obtaining real-time physiological data relating to a sleep state of a user; and controlling delivery of one or more active compounds based, at least in part, on a determined and/or a desired sleep state of a user, wherein the one or more active compounds influence, manage, change or control a mood state of the user.

### FIELD

The present specification relates to controlling or influencing a sleep state of user, for example by controlling or influencing one or more of: preparation for sleep, sleep maintenance, and preparation for waking.

### BACKGROUND

The use of sensors to monitor a person's sleep state is known. There remains a need for further developments in the field of controlling or influencing sleep states.

### SUMMARY

In a first aspect, this specification describes a system comprising: an input module for obtaining real-time physiological data relating to a sleep state of a user; a control module for determining delivery of one or more active compounds based, at least in part, on a determined or a desired sleep state of the user, wherein the one or more active compounds influence, manage, change or control a mood state of said user; and an output module for communicating with one or more dispensers for delivery of said one or more active compounds.

The control module may be configured to influence manage, change or control the sleep state of the user by managing a user environment dependent, at least in part, on a determined stage of a sleep cycle of the user.

The input module may be for obtaining environment data. The environmental data may comprise data relating to light and/or sound.

The system may further comprise a pillow, where some or all of said environment data are obtained from one or more sensors of the pillow.

The system may further comprise one or more dispensers for delivering said one or more active compounds under the control of said output module. The one or more dispensers may deliver said one or more active compounds into a room including said user. The one or more dispensers may include one or more dispensers of the pillow described above.

In a second aspect, this specification describes a method comprising: obtaining real-time physiological data relating to a sleep state of a user; and controlling delivery of one or more active compounds based, at least in part, on a determined and/or a desired sleep state of a user, wherein the one or more active compounds influence, manage, change or control a mood state of the user.

The sleep state of the user may be influenced, managed, changed or controlled, at least in part, by managing a user environment dependent, at least in part, on a determined stage of a sleep cycle of the user.

The method may further comprise determining whether the user is entering or leaving a sleep stage and managing said environment accordingly.

The method may further comprise obtaining environment data, such as data relating to light and/or sound.

Some or all of said one or more active compounds may be delivered by dispensers of a pillow. Alternatively, or in addition, some or all of said environment data may be obtained from one or more sensors of a/the pillow.

In a third aspect, this specification describes computer-readable instructions which, when executed by a computing apparatus, cause the computing apparatus to perform (at least) any method as described herein (including the method of the second aspect described above).

In a fourth aspect, this specification describes a computer-readable medium (such as a non-transitory computer-readable medium) comprising program instructions stored thereon for performing (at least) any method as described herein (including the method of the second aspect described above).

In a fifth aspect, this specification describes an apparatus comprising: at least one processor; and at least one memory including computer program code which, when executed by the at least one processor, causes the apparatus to perform (at least) any method as described herein (including the method of the second aspect described above).

In a sixth aspect, this specification describes a computer program comprising instructions for causing an apparatus to perform at least: obtaining real-time physiological data relating to a sleep state of a user; and controlling delivery of one or more active compounds based, at least in part, on a determined and/or a desired sleep state of a user, wherein the one or more active compounds influence, manage, change or control a mood state of the user.

### BRIEF DESCRIPTION OF THE DRAWINGS

Example embodiments will now be described, by way of example only, with reference to the following schematic drawings, in which:

FIG. 1 is a hypnogram illustrating a sleep state of a person as a function of time, in accordance with an example embodiment;

FIG. 2 is a block diagram of a system in accordance with an example embodiment;

FIG. 3 is a flow chart showing an algorithm in accordance with an example embodiment;

FIG. 4 is a block diagram of a system in accordance with an example embodiment;

FIG. 5 is a block diagram of a system in accordance with an example embodiment;

FIG. 6 is a block diagram of a system in accordance with an example embodiment;

FIG. 7 illustrates a system including a pillow in accordance with an example embodiment;

FIG. 8 illustrates a system including a pillow in accordance with an example embodiment;

FIG. 9 illustrates a system in accordance with an example embodiment;

FIG. 10 is a flow chart showing an algorithm in accordance with an example embodiment;

FIG. 11 is a block diagram of an aerosol generating device in accordance with an example embodiment;

FIG. 12 is a block diagram of a system in accordance with an example embodiment;

FIG. 13 is a block diagram showing an aerosol delivery mechanism in accordance with an example embodiment; and

FIG. 14 is a block diagram, of a processing system that may be used to implement one or more of the example embodiments.

### DETAILED DESCRIPTION

As used herein, the term “delivery mechanism” is intended to encompass systems that deliver a substance to a user, and includes:
non-combustible aerosol provision systems that release compounds from an aerosolisable material without combusting the aerosolisable material, such as electronic cigarettes, tobacco heating products, and hybrid systems to generate aerosol using a combination of aerosolisable materials; and
articles comprising aerosolisable material and configured to be used in one of these non-combustible aerosol provision systems.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

In some embodiments, the substance to be delivered comprises an active substance (sometimes referred to herein as an active compound).

The active substance as used herein may be a physiologically active material, which is a material intended to achieve or enhance a physiological response. The active substance may for example be selected from nutraceuticals, nootropics, psychoactives, or digiceutical or other technical/electronic devices that may induce a physiological response, such as vagus nerve stimulation (VGS). The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical. In one embodiment, the active substance is a legally permissible recreational drug.

In some embodiments, the active substance comprises nicotine. In some embodiments, the active substance comprises caffeine, melatonin or vitamin B12.

As noted herein, the active substance may comprise one or more constituents, derivatives or extracts of cannabis, such as one or more cannabinoids or terpenes.

As noted herein, the active substance may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens

In some embodiments, the active substance comprises or is derived from one or more botanicals or constituents, derivatives or extracts thereof and the botanical is tobacco.

In some embodiments, the active substance comprises or derived from one or more botanicals or constituents, derivatives or extracts thereof and the botanical is selected from eucalyptus, star anise, cocoa and hemp.

In some embodiments, the active substance comprises or derived from one or more botanicals or constituents, derivatives or extracts thereof and the botanical is selected from rooibos and fennel.

Aerosolisable material, which also may be referred to herein as aerosol generating material, is material that is capable of generating aerosol, for example when heated, irradiated or energized in any other way. Aerosolisable material may, for example, be in the form of a solid, liquid or gel which may or may not contain nicotine and/or flavourants.

The aerosol-generating material may be an “amorphous solid”. In some embodiments, the amorphous solid is a “monolithic solid”. The aerosol-generating material may be non-fibrous or fibrous. In some embodiments, the aerosol-generating material may be a dried gel. The aerosol-generating material may be a solid material that may retain some fluid, such as liquid, within it. In some embodiments the retained fluid may be water (such as water absorbed from the surroundings of the aerosol-generating material) or the retained fluid may be solvent (such as when the aerosol-generating material is formed from a slurry). In some embodiments, the solvent may be water.

The aerosol-generating material may comprise one or more active substances and/or flavours, one or more aerosol-former materials, and optionally one or more other functional material.

The aerosol-former material may comprise one or more constituents capable of forming an aerosol. In some embodiments, the aerosol-former material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, colouring agents, preservatives, binders, fillers, stabilizers, and/or antioxidants.

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

A mood state of a person (or user) may depend on a number of environmental factors such as work, exercise, gaming or any activity. Example embodiments described herein provide arrangements for determine current user mood states, predicting future mood states and managing, influencing or controlling mood states.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

Generally, a person will experience multiple sleep cycles per night. For example, a person may experience 4 to 6 sleep cycles per night. Each sleep cycle comprises four distinct stages, hereinafter referred to as stage 1, stage 2, stage 3 and stage 4.

Stage 1: light non-Rapid Eye Movement (REM) sleep. In this state, the person's heart rate, breathing, eye movements and brain waves slow down. Stage 1 typically lasts for about 1 to 5 minutes.

Stage 2: deeper non-REM sleep. In this state, the muscles relax further, eye movements stop and body temperature drops. The first occurrence of this stage typically lasts for about 25 minutes. Subsequent occurrences are generally longer.

Stage 3: deepest non-REM sleep. In this state, heart rate, breathing and brain waves become regular. Generally, this state is experienced during the first half of the night. Subsequent occurrences are generally shorter.

Stage 4: REM sleep. In this state, a person's eye move rapidly from side to side, breathing quickens and becomes more erratic, blood pressure and heart rate become less regular. This state typically lasts for approximately 10 minutes during the first sleep cycle, increasing in length as the night progresses. In the final cycle, it can last up to 1 hour.

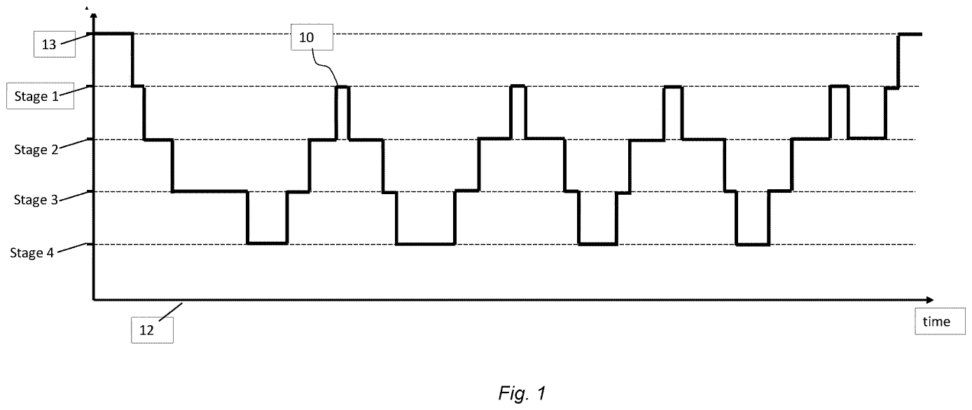

FIG. 1 is a hypnogram illustrating a sleep state 10 of a person as a function of time 12, in accordance with an example embodiment. The person transitions from wakefulness 13 then cycles through stages 1, 2, 3 and 4 four times, returns to stage 2 and finally transitions to stage 1 before returning to wakefulness 13.

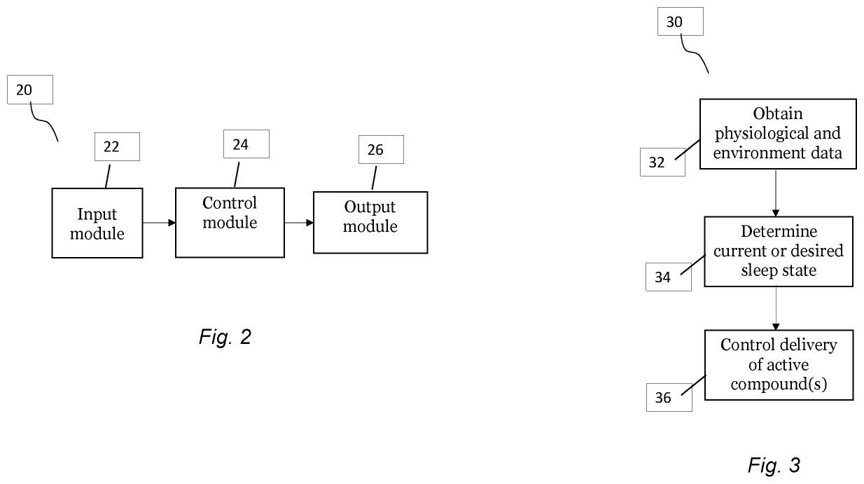

FIG. 2 is a block diagram of a system, indicated generally by reference numeral 20, in accordance with an example embodiment. The system 20 comprises an input module 22, a control module 24 and an output module 26. The input module 22 receives data, such as physiological data relating to a sleep state of a user and environment data (such as temperature, lighting levels, noise levels etc.). The control module 24 seeks to provide at least some control over the user's sleep state, for example by determining delivery of one or more active compounds (sometimes referred to herein as active substances) that are known to influence, change or control a mood state of the user. As discussed further below, the control module may determine the delivery of active compound(s) based, at least in part, on a determined or desired sleep state of the user (e.g. based on a determine stage or state of a sleep cycle of the user). The output module 26 may comprise one or more dispensers or a communication device for communicating with one or more remote dispensers. The dispenser(s) are configured to deliver the one or more active compounds into the room i.e. near the user. The dispensers may deliver the one or more active compounds in the form of an aerosol, but this is not essential to all example embodiments; for example one or more active compounds may be delivered as a mist or a spray.

FIG. 3 is a flow chart showing an algorithm, indicated generally by reference numeral 30, in accordance with an example embodiment. The algorithm 30 may be implemented using the system, 20 described above.

The algorithm starts at operation 32, where physiological data relating to a sleep state of a user and environment data relating to a location/room (generally the room in which the user and/or system is located). The data may be (or may be derived from) the data obtained by the input module 22 described above. As discussed further below, at least some of data may be obtained by one or more sensors, thereby providing real-time physiological data.

At operation 34, the current or desired sleep state is determined. The operation 34 may be implemented by the control module 24 of the system 20. There are many arrangements by which sleep cycle information may be obtained. These include use of one or more of: electrocardiogram (ECG or EKG) data, electroencephalogram (EEG) data, temperature, oxygen usage, and eye movement data (e.g. using cameras to track eye movement).

At operation 36, delivery of one or more active compounds (to the user) is controlled. The active compounds are suitable for managing, influencing, changing or controlling a mood state of the user. The operation 36 may include determining/selecting one or more active compounds to be delivered to the user (e.g. in the form of an aerosol, mist or spray). The operation 36 may include timing of delivery of selected compound(s) (e.g. a delivery start time and a deliver duration). The operation 36 may include determining a delivery dose of selected active compound(s). Selection of active compound(s) may be based on a-prior knowledge of their effects on user mood or sleep state. Thus, deliver of the selected compound(s) can be defined to seek to change or control the sleep state of the user. The operation 36 may be implemented by the control module 24 of the system 20.

There are many active compounds, or combinations of active compounds, that may be delivered under the control of the algorithm 20. Examples include melatonin (e.g. to aid sleep), caffeine (e.g. to aid focus or alertness or to provide energy) and/or cannabidiol (CBD) (e.g. to aid relaxation). The skilled person will be aware of many other compounds (or combinations of compounds) that could be used (including other active compounds mentioned herein).

The control module 24 may comprise a look up table providing the intended effect of each active compound available to the system such as stress relief etc. The look up table may further comprise details of the advised delivery time and delivery duration of each active compound in order to achieve the intended effect. The look up table may therefore be used in the selection of active compounds in the operation 36 of the algorithm 30 in order to meet a need and/or in determining the delivery parameters of a selected active compound to meet a need.

The algorithm 30 may be iterative, so that the control of the delivery of active compound(s) is updated as the determined or desired sleep state changes over time or as delivery requirements in order to achieve a desired sleep state changes.

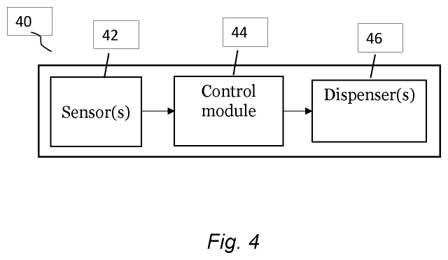

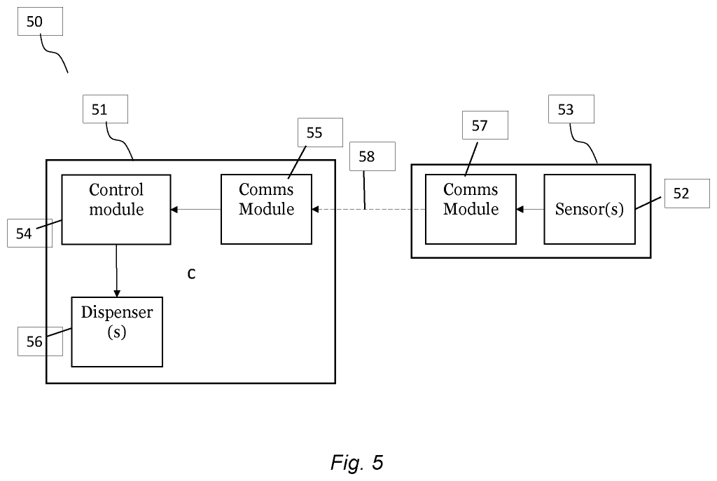

FIG. 4 is a block diagram of a system, indicated generally by the reference numeral 40, in accordance with an example embodiment. The system 40 may be part of a single integrated device used by the user whilst sleeping. For example (as discussed further below), the device may form part of a head support such as a pillow or a head rest in a chair. The system 40 comprises one or more sensor(s) 42, a control module 44 and one or more dispenser(s) 46. The sensor(s) 42 may obtain real-time physiological data relating to a sleep state of a user. The sensor(s) may also obtain data relating to the environment at a location or room; typically where the user is located. The sensor(s) 42 may therefore be (or form part of) the input module 22 described above. The control module 44 may be an example implementation of the control module 24 described above. The dispenser(s) 46 may be configured to deliver the one or more active compounds for example into a room including the users. Some or all of the one or more active compounds may be delivered in the form of an aerosol. The dispenser(s) 46 may include one or more aerosol generating devices. As discussed further below, in some embodiments, the one or more active compounds comprise a liquid material and the dispenser(s) 46 comprise a heater configured to heat the one or more active compounds to generate an aerosol. Moreover, as discussed further below, the dispenser(s) 46 may have access to multiple active compounds and include a mechanism for selecting between active compounds for delivery.

The sensor(s) described herein (such as the sensor(s) 42) may include any sensor that transduces a physiological trait or characteristic into an electrical signal that can be processed to determine, measure or track that particular trait or characteristic. This includes sensors that are cameras, microphones, electrophysiological sensors (EEG, ECG etc.), temperature sensors etc.

FIG. 5 is a block diagram of a system, indicated generally by reference numeral 50, in accordance with an example embodiment. The system 50 comprises first and second devices; labelled 51 and 53 respectively. Either device may form part of a head support used by the user when sleeping; for example the head support may be a pillow or a head rest in a chair. Either device may be remote from the user. The first device 51 comprises a control module 54, one or more dispenser(s) 56 and a communications module 55. The second device 53 comprises one or more sensor(s) 52 and a communications module 57. The control module 54 may be an example implementation of the control module 24 described above. The dispenser(s) 56 may be the same or similar to the dispenser(s) 46 described above. The sensor(s) 52 may be the same or similar to the sensor(s) 42 described above. The communications modules 55 and 57 are configured to transfer data from the second device 53 to the first device 51 via datalink 58. The data may be (or may include) the data obtained by the sensor(s) 52. The datalink 58 may include a cable or wireless datalinks.

The system 50 may be used in an example implementation of the algorithm 30 described above.

The sensor(s) 52 may be used to obtain physiological data relating to a sleep state of a user and, for example, environment data relating to a location/room (thereby implementing operation 32 of the algorithm 30). The data obtained by the sensors 52 may be sent to the control module 54 via the communications modules 55 and 57 (possibly following pre-processing of the sensor data).

The control module 54 may be used to determine the current or desired sleep state (thereby implementing the operation 34 of the algorithm 30).

The one or more active compounds may be delivered to the user using the dispenser(s) 56 (thereby implementing the operation 36 of the algorithm 30).

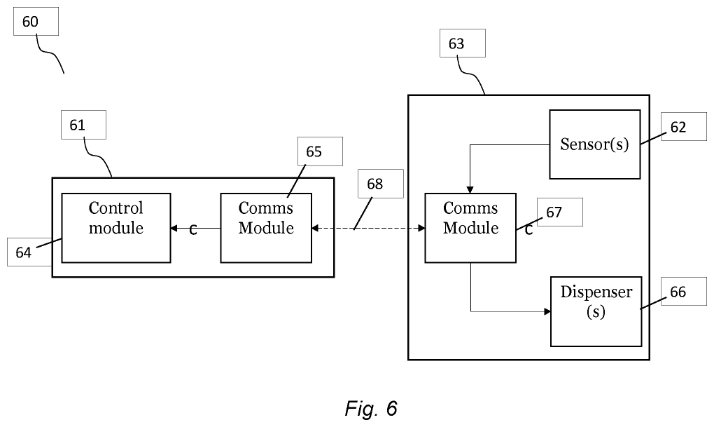

FIG. 6 is a block diagram of a system, indicated generally by reference numeral 60, in accordance with an example embodiment. The system 60 comprises first and second devices; labelled 61 and 63 respectively. Either device may form part of a head support used by the user when sleeping; for example the head support may be a pillow or a head rest in a chair. Either device may be remote from user. The first device 61 comprises a control module 64 and a communications module 65. The second device 63 comprises one or more sensor(s) 62, one or more dispenser(s) 66 and a communications module 67. The control module 64 may be an example implementation of the control module 24 described above. The dispenser(s) 66 may be the same or similar to the dispenser(s) 46 described above. The sensor(s) 62 may be the same or similar to the sensor(s) 42 described above. The communications modules 65 and 67 are configured to transfer data to and from the first device 61 and second device 63 via a two-way datalink 68. The data may be the data obtained by the sensor(s) 62 and a determined or a desired sleep state of the user or one or more active compounds to deliver. The datalink 68 may include a cable or wireless datalinks.

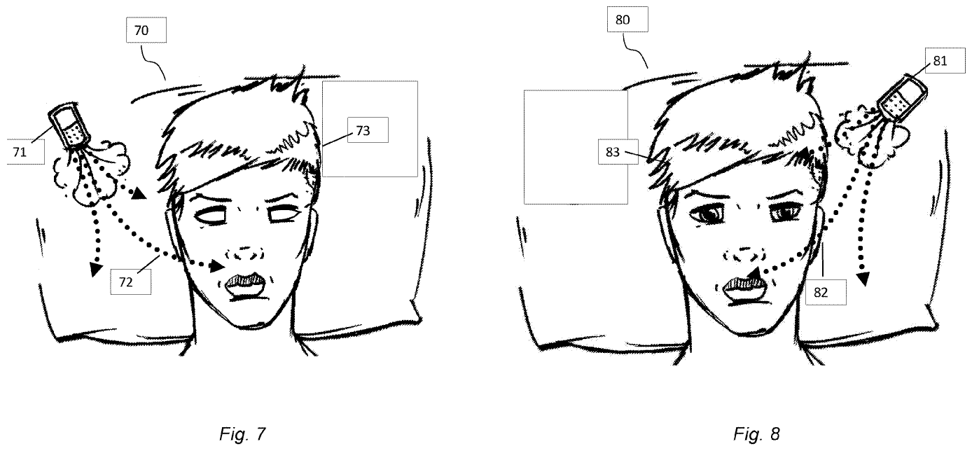

FIG. 7 illustrates a system 71 in accordance with an example embodiment. The system 71 may be part of a pillow 70. The system 71 comprises sensor(s), a control module and dispenser(s) and may be the same as or similar to the device 40 described above. The sensor(s) may be the same or similar to sensor(s) 42 described above. The control module may be an example implementation of the control module 24 described above. The dispenser(s) may be the same or similar to the dispenser(s) 46 described above.

In one example embodiment, the pillow 70 may be used to help a user 73 in a sleep state transition to wakefulness. The sensor(s) collects real-time physiological data (such as one or more of: ECG or EKG data, EEG data, temperature, oxygen usage, and eye movement data) relating to the sleep state of the user (for example the current sleep stage of the user) and environment data (for example the light level and/or sound types). The control module may decide that the user should be progressively transitioned from one sleep stage (e.g. stage 3) to other sleep stages (e.g. stage 2, then stage 1) and finally to wakefulness. The control module of the system 71 instructs the dispenser(s) to deliver one or more active compounds at specific times and for specific durations to achieve the desired sleep state transitions. The dispenser(s) of the system 71 deliver the active compounds as an aerosol, mist or spray 72; which are inhaled by the user 73.

FIG. 8 illustrates a system 81 in accordance with an example embodiment. The system 81 may be part of a pillow 80. The system 81 comprises sensor(s), a control module and dispenser(s) and may be the same as or similar to device 40 described above. The sensor(s) may be the same or similar to sensor(s) 42 described above. The control module may be an example implementation of the control module 24 described above. The dispenser(s) may be the same or similar to the dispenser(s) 46 described above. In an example embodiment, the pillow 80 may be used to help a user 83 in a wakefulness state transition to sleep. The sensor(s) collect real-time physiological data (such as one or more of: ECG or EKG data, EEG data, temperature, oxygen usage, and eye movement data) relating to the sleep state of the user (for example wakefulness) and environment data (for example the light level and/or sound types). The control module may decide the user should be progressively transitioned from wakefulness to stage 1, stage 2, stage 3 and then stage 4. The control module instructs the dispenser(s) to deliver one or more active compounds at specific times and for specific durations to achieve the desired sleep state transitions. The dispenser(s) deliver the active compounds as an aerosol, mist or spray 82; which are inhaled by the user 83.

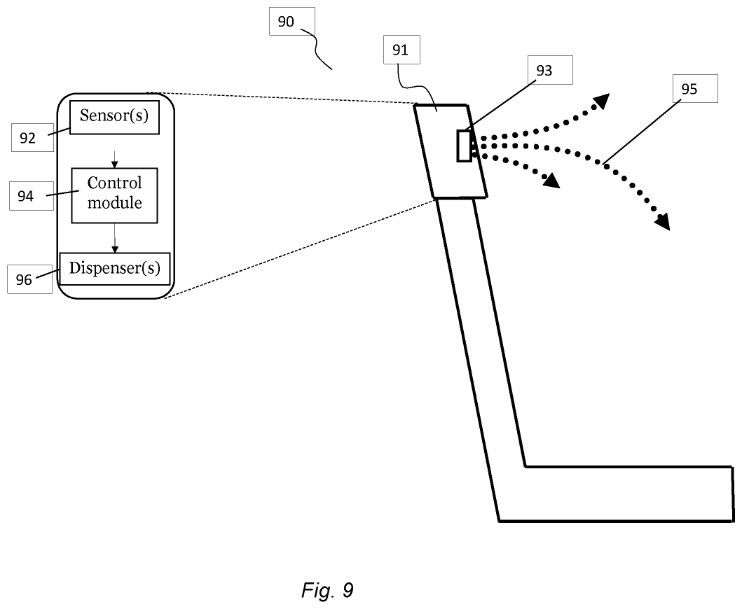

FIG. 9 illustrates a system 93 in accordance with an example embodiment. The system 93 may be part of a headrest 91 on a chair 90. The system 93 comprises sensor(s) 92, a control module 94 and one or more dispenser(s) 96; shown in FIG. 9 in a magnified view. The sensor(s) 92 may be the same or similar to the sensor(s) 42 described above. The control module 94 may be an example implementation of the control module 24 described above. The dispenser(s) 96 may be the same or similar to the dispenser(s) 46 described above.

By way of example, the chair 90 may be used to help a user (not shown) stay awake and alert; for example whilst driving a vehicle or working in an office chair. The sensor(s) 92 collect real-time physiological data relating to the wakefulness state of the user and environment data (for example light level and/or sound types). The control module 94 may monitor signs of tiredness of the user and decide to intervene to help the user remain awake (or maintain a state of wakefulness). The control module 94 instructs the dispenser(s) 96 to deliver one or more active compounds at specific times and for specific duration to keep the user awake and alert. The dispenser(s) deliver the active compounds as an aerosol, mist or spray 95; which are inhaled by the user.

Alternatively, or in addition, the chair 90 may be used to help a user to enter a sleep mode (or to enter a different stage of a sleep mode). In this embodiment, the control module 94 instructs the dispenser(s) 96 to deliver one or more active compounds at specific times and for specific duration to control the sleep state of the user.

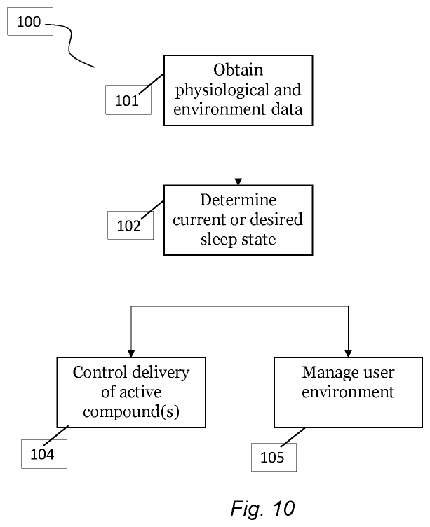

FIG. 10 is a flow chart showing an algorithm, indicated generally by reference numeral 100, in accordance with an example embodiment. The algorithm 100 may be implemented using any of the systems or devices 20, 40, 50, 60, 71, 81 or 93 described above.

The algorithm starts at operation 101, where physiological data relating to a sleep state of a user and environment data relating to a location/room (generally the room in which the user and/or system is located) are obtained. The data may be (or may be derived from) data obtained by an input module (e.g. the input module 22 described above). At least some of data may be obtained from one or more sensors, thereby providing real-time physiological data (such as one or more of: ECG or EKG data, EEG data, temperature, oxygen usage, and eye movement data). At least some of the sensors providing physiological data and/or environment data form part of a pillow, as discussed above. Example sensors include thermal sensors, non-thermal sensors, galvanic skin response (GSR) sensor, eye tracking/dilation sensors, transdermal optical imaging (TOI) sensors, and other sensors to detect brain activity etc.

At operation 102, the current or desired sleep state is determined. The operation 102 may be implemented by the control module 24 of the system 20 (or a similar control module).

At operation 104, delivery of one or more active compounds (to the user) is controlled. The active compounds are suitable for influencing, managing, changing or controlling a mood state of the user. The operation 104 may include determining/selecting one or more active compounds to be delivered to the user. The operation 104 may include timing of delivery of selected compound(s) (e.g. a delivery start time and a deliver duration). The operation 104 may include determining a delivery dose of selected active compound(s). Selection of active compound(s) may be based on a-prior knowledge of their effects on user mood or sleep state. Thus, deliver of the selected compound(s) can be defined to seek to manage, influence, change or control the sleep state of the user. The operation 104 may be implemented by the control module 24 of the system 20 (or a similar control module).

At operation 105, the sleep state of the user is influenced, managed, changed or controlled by managing the user environment dependent, at least in part, on a determined stage of a sleep cycle of the user. The operation 105 may include switching on one or more lights or adjusting light levels at a particular time and for a particular duration. The operation 105 may include emitting sounds at particular times and durations. Selection of lights or sounds may be based on a-priori knowledge of their effects on user mood. Thus, delivery of the lights or sounds may change or control the mood state of the user. The operation 105 may be implemented by the control module 24 of the system 20 (or a similar control module).

The algorithm 100 may be iterative, so that the control of the delivery of active compound(s) and/or the management of the user environment is updated as the determined or desired sleep state changes over time or as requirements in order to achieve a desired sleep state changes.

In some embodiments, the sleep state of the user is managed, changed or controlled by managing the user environment and is further dependent on whether the user is entering or leaving a sleep stage (for example, if the user is awake and is beginning to fall asleep). In another example embodiment, the user may be asleep and beginning to wake up before a predetermined time. For example, the light levels or sound types, frequencies and/or level may be adjusted to help the user stay awake or remain asleep.

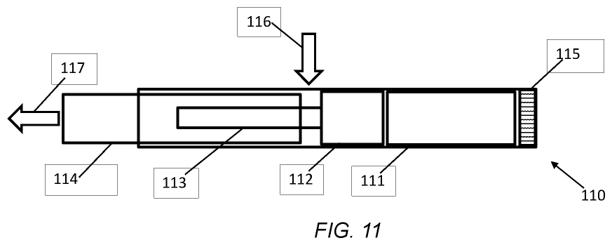

FIG. 11 is a block diagram of an aerosol generating device, indicated generally by the reference numeral 110, in accordance with an example embodiment. The aerosol generating device 110 may be used as, or form part of, one or more of the dispensers described above (such as the dispensers 46, 56, 66 or 96).

The aerosol generating device 110 comprises a battery 111, a control circuit 112, a heater 113 and a consumable 114. The device also includes a connector 115 (such as a USB connector). The connector 115 may enable connection to be made to a power source for charging the battery 111, for example under the control of the control circuit 112. The control circuit 112 may form part of (or being under the control of) one of the control modules described above (such as the control modules 24, 44, 54, 64 and 94).

In the use of the device 110, the heater 113 is inserted into the consumable 114, such that the consumable may be heated to generate an aerosol for the user. In the use of the device 110, air is drawn into the device through an air inlet as indicated by arrow 116, then passes through the consumable, delivering the aerosol to the user as indicated by the arrow 117.

The aerosol generating device 110 is provided by way of example only. Many alternative aerosol generating devices may be used in example implementations of the principles described here. For example, the device 110 may be replaced within a vaping device in which an aerosol generating material (e.g. a liquid) is heated to generate the aerosol. Moreover, the aerosol generating device 110 may have access to multiple active compounds and include a mechanism for selecting between active compounds for delivery, as discussed further below. Such multiple active compounds could be mixed into bespoke formulations, for example based on past used experience.

Furthermore, the aerosol generating device 110 may be replaced with an alternative device for delivering active compounds in the form of a mist or spray. Other suitable arrangements will be apparent to those of ordinary skill in the art.

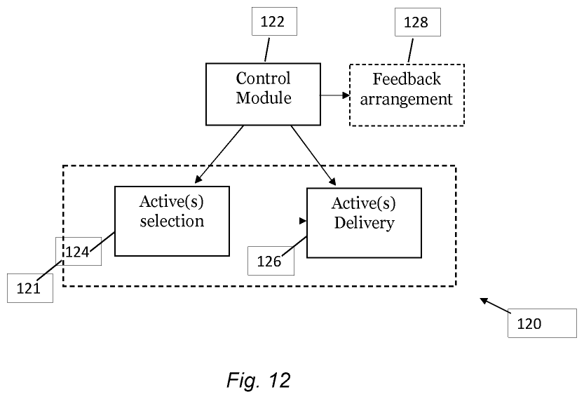

FIG. 12 is a block diagram of a system, indicated generally by the reference numeral 120, in accordance with an example embodiment.

The system 120 includes a dispenser 121 may be used as, or form part of, one or more of the dispensers described above (such as the dispensers 46, 56, 66 or 96). The system 120 also includes a control module 122 and a feedback arrangement 128. In the example system 120, the dispenser 121 comprises an active compound(s) selection module 124 and an active compound(s) delivery module 126. The delivery module 126 may comprise an aerosol delivery mechanism, as discussed above.

In the system 120, the control module 122 controls the dispenser 121. For example, in the system 120, the control module 122 may control the selection of one or more active components for delivery (by controlling the active compound(s) selection module 124) and may also control the delivery of the selected compound(s) (by controlling the active compound(s) delivery module 126).

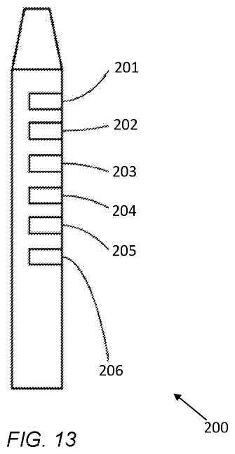

FIG. 13 is a block diagram showing an aerosol delivery mechanism, indicated generally by the reference numeral 200, in accordance with an example embodiment.

In broad outline, the device 200 is configured to generate an aerosol for delivery to a user from at least one aerosolisable material received within the device 200. Herein an aerosolisable material includes any material that may be aerosolised. In the examples discussed herein the aerosol provision device 200 is configured to receive a plurality of aerosolisable materials, where each aerosolisable material is housed in or forms a consumable, e.g., the consumable may be a container housing the aerosolisable material. The aerosol provision device 200 is configured to receive at least a first consumable 201 and a second consumable 202, and the device 200 may also be configured to receive further consumables 203, 204, 205 and 206. Herein, reference is made to the device 200 receiving consumables 201 to 206; however, it should be appreciated that device 200 more generally receives a plurality of aerosolisable materials. In some implementations, the aerosolisable materials may be provided detached from one another (e.g., as separate consumables as described herein) or may be provided on a common substrate as a single consumable to be received in the device 200.

The aerosol delivery mechanism 200 may be configured to recognise the identity and position of consumables 201 to 206 received in the device 200 and may transmit data indicating the identity and position of consumables received in the device 200 (e.g. to a control module, such as the control module 24, 44, 54, 64 or 94 described above). The consumables 201 to 206 may, for example, comprise radio frequency identification (RFID) tags that may be used for identification purposes.

The aerosol delivery mechanism 200 allows for a usage session which is appropriate for the consumables received within the device 200 to be implemented. Appropriate settings may be applied to the aerosol delivery mechanism 200 depending on the consumables inserted and depending on the contextual environment of the user (e.g. depending on a determined or predicted user need). Herein, reference is made to example devices transmitting data regarding the identity and position of consumables or, more generally, aerosolisable materials received in the device. It should be appreciated that in some implementations, a device may recognise the identity and/or position of consumables/materials received in the device and a controller or the like in the device may use the identity and position data to provide instructions to the device for producing an aerosol based on the identity and/or position of consumables/materials received in the device.

The device 200 comprises means for receiving at least the first consumable 201 for containing a first aerosolisable material, and for receiving the second consumable 202 for containing a second material. In some examples, the device is configured to receive further consumables, such as third 203, fourth 204, fifth 205, and sixth 206 consumables for containing third, fourth, fifth and sixth aerosolisable materials respectively. In other examples, the device 200 may be configured to receive any number, two or more, of consumables.

Aerosol is generated by the device 200 from at least the first consumable 201 containing first aerosolisable material. The first consumable 201 is in fluidic contact with a central aperture (for example via a value or flow device, not shown), and air flowing in through one or more air inlets mixes with aerosol generated from the first consumable 201 to generate a flow of aerosol. The aerosol flow is drawn towards the outlet for delivery to the user. In some examples, air flowing from the air inlets to the mouthpiece may pass through each consumable or aerosolisable material received in the device sequentially. That is, each of the consumables or aerosolisable materials in the device may be located on the same air flow path between the air inlets and the mouthpiece. In other examples, there may be multiple branches for air flowing from the air inlet/s towards the outlet. For example, a plurality of branches may be provided and each branch of the plurality of branches may pass through one or more of the consumables or aerosolisable materials. There may be one branch for each of the consumables or aerosolisable materials, or each air flow path may pass through more than one of the consumables or aerosolisable materials. In some examples, where there are multiple air flow branches there may be a branch which does not pass through a consumable or aerosolisable material. Where there are multiple air flow branches the branches may join, in an admixing chamber or the like, prior to aerosol flowing to the mouthpiece.

The second consumable 202 may also produce aerosol which mixes with the aerosol generated from the first consumable 201 before the aerosol reaches the outlet. For example, the second consumable 202 may produce a flavoured aerosol. Additionally or alternatively, one or more properties of the aerosol generated from the first consumable 201 may be modified by material contained by the second consumable 202 and, optionally, by material contained by one or more further consumables 203, 204, 205, etc. received within the device.

In some example embodiments, the aerosolisable materials may be liquids or gels; however this is not essential to all example embodiments.

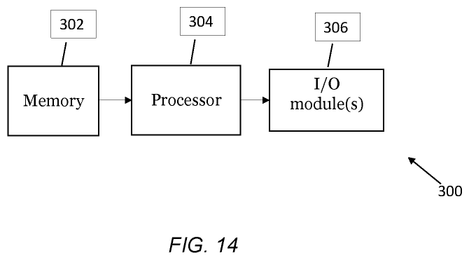

FIG. 14 is a block diagram of a processing system, indicated generally by the reference numeral 300, that may be used to implement one or more of the example embodiments described previously. The processing system 300 may, for example, be (or may include) the apparatus referred to in the claims below.

The processing system 300 may have a processor 304, a memory 302 coupled to the processor (e.g. comprising a random access memory (RAM) and/or a read only memory (ROM)). The processing system 300 may also comprise one or more input/output (I/O) modules 306, such as one or more user interface modules.

The memory 302 may comprise code which, when executed by the processor 304 implements aspects of the methods and algorithms described herein.

Aspects of the subject matter described herein are set out in the following numbered clauses:

1. A system comprising:
an input module for obtaining real-time physiological data relating to a sleep state of a user;
a control module for determining delivery of one or more active compounds based, at least in part, on a determined or a desired sleep state of the user, wherein the one or more active compounds influence, manage, change or control a mood state of said user; and
an output module for communicating with one or more dispensers for delivery of said one or more active compounds.

2. The system according to clause 1, wherein the control module is configured to influence manage, change or control the sleep state of the user by managing a user environment dependent, at least in part, on a determined stage of a sleep cycle of the user.

3. The system according to clause 1 or clause 2, wherein the input module is for obtaining environment data.

4. The system according to clause 3, wherein the environmental data comprises data relating to light and/or sound.

5. The system according to clause 3 or clause 4, further comprising a pillow, where some or all of said environment data are obtained from one or more sensors of the pillow.

6. The system according to any one of the preceding clauses, further comprising the one or more dispensers for delivering said one or more active compounds under the control of said output module.

7. The system according to clause 6, further comprising a pillow, where some or all of said environment data are obtained from one or more sensors of the pillow, and wherein said one or more dispensers include one or more dispensers of the pillow.

8. The system according to any one of the preceding clauses, wherein the one or more dispensers deliver said one or more active compounds into a room including said user.

9. The system according to clause 8, further comprising a pillow, where some or all of said environment data are obtained from one or more sensors of the pillow, and wherein said one or more dispensers include one or more dispensers of the pillow.

10. A method comprising:
obtaining real-time physiological data relating to a sleep state of a user; and
controlling delivery of one or more active compounds based, at least in part, on a determined and/or a desired sleep state of a user, wherein the one or more active compounds influence, manage, change or control a mood state of the user.

11. The method according to clause 10, wherein the sleep state of the user is influenced, managed, changed or controlled, at least in part, by managing a user environment dependent, at least in part, on a determined stage of a sleep cycle of the user.

12. The method according to clause 10 or clause 11, further comprising determining whether the user is entering or leaving a sleep stage and managing said environment accordingly.

13. The method according to any one of clauses 10 to 12, further comprising obtaining environment data.

14. The method according to clause 13, wherein the environment data comprises data relating to light and/or sound.

15. The method according to any one of clauses 10 to 14, wherein some or all of said one or more active compounds are delivered by dispensers of a pillow.

16. The method according to any one of clauses 10 to 15, where some or all of said environment data are obtained from one or more sensors of a/the pillow.

17. A computer program comprising instructions for causing an apparatus to perform at least the following:
obtaining real-time physiological data relating to a sleep state of a user; and
controlling delivery of one or more active compounds based, at least in part, on a determined and/or a desired sleep state of a user, wherein the one or more active compounds influence, manage, change or control a mood state of the user.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A system comprising:
an input module for obtaining real-time physiological data relating to a sleep state of a user;
a control module for determining delivery of one or more active compounds based, at least in part, on a determined or a desired sleep state of the user, wherein the one or more active compounds influence, manage, change or control a mood state of said user; and
an output module for communicating with one or more dispensers for delivery of said one or more active compounds.

2. The system as claimed in claim 1, wherein the control module is configured to influence manage, change or control the sleep state of the user by managing a user environment dependent, at least in part, on a determined stage of a sleep cycle of the user.

3. The system as claimed in claim 1, wherein the input module is for obtaining environment data.

4. The system as claimed in claim 3, wherein the environmental data comprises data relating to light and/or sound.

5. The system as claimed in claim 3, further comprising a pillow, where some or all of said environment data are obtained from one or more sensors of the pillow.

6. The system as claimed in claim 1, further comprising the one or more dispensers for delivering said one or more active compounds under the control of said output module.

7. The system as claimed in claim 6, further comprising a pillow, where some or all of said environment data are obtained from one or more sensors of the pillow, and wherein said one or more dispensers include one or more dispensers of the pillow.

8. The system as claimed in claim 1, wherein the one or more dispensers deliver said one or more active compounds into a room including said user.

9. The system as claimed in claim 8, further comprising a pillow, where some or all of said environment data are obtained from one or more sensors of the pillow, and wherein said one or more dispensers include one or more dispensers of the pillow.

10. A method comprising:
obtaining real-time physiological data relating to a sleep state of a user; and
controlling delivery of one or more active compounds based, at least in part, on a determined and/or a desired sleep state of a user, wherein the one or more active compounds influence, manage, change or control a mood state of the user.

11. The method as claimed in claim 10, wherein the sleep state of the user is influenced, managed, changed or controlled, at least in part, by managing a user environment dependent, at least in part, on a determined stage of a sleep cycle of the user.

12. The method as claimed in claim 10, further comprising determining whether the user is entering or leaving a sleep stage and managing said environment accordingly.

13. The method as claimed in claim 10, further comprising obtaining environment data.

14. The method as claimed in claim 13, wherein the environment data comprises data relating to light and/or sound.

15. The method as claimed in claim 10, wherein some or all of said one or more active compounds are delivered by dispensers of a pillow.

16. The method as claimed in claim 10, where some or all of said environment data are obtained from one or more sensors of a/the pillow.

17. A computer program comprising instructions for causing an apparatus to perform at least the following:
obtaining real-time physiological data relating to a sleep state of a user; and
controlling delivery of one or more active compounds based, at least in part, on a determined and/or a desired sleep state of a user, wherein the one or more active compounds influence, manage, change or control a mood state of the user.
